(19) United States
(12) Patent Application Publication
(10) Pub. No.: US 2024/0423500 A1
(43) Pub. Date: Dec. 26, 2024

(54) ELECTRONIC DEVICE, ARM SWING ANALYSIS METHOD, AND STORAGE MEDIUM

(71) Applicant: CASIO COMPUTER CO., LTD., Tokyo (JP)

(72) Inventors: Keisuke SHIMADA, Tokyo (JP); Hiroyasu IDE, Tokyo (JP); Yuto YAMANAKA, Tokyo (JP); Takashi SAKURAI, Tokyo (JP); Masaaki SASAKI, Tokyo (JP); Takehiro AIBARA, Tokyo (JP); Takashi NIIMURA, Tokyo (JP); Tomoharu YAMAGUCHI, Tokyo (JP); Yoshitsugu MANABE, Tokyo (JP)

(73) Assignee: CASIO COMPUTER CO., LTD., Tokyo (JP)

(21) Appl. No.: 18/752,960

(22) Filed: Jun. 25, 2024

(30) Foreign Application Priority Data
Jun. 26, 2023 (JP) 2023-103950

(51) Int. Cl. A61B 5/11; A61B 5/00

(52) U.S. Cl. CPC A61B 5/1114; A61B 5/1121; A61B 5/1123; A61B 5/458; A61B 5/681; A61B 5/7278; A61B 2562/0219

(57) ABSTRACT

An electronic device includes at least one processor that identifies a cyclic change related to an arm swing of a user from a three-dimensional acceleration measured by a measurer. The at least one processor defines a two-dimensional plane containing a plane of the arm swing of the user from the three-dimensional acceleration measured at a wrist of the user, detects the cyclic change as a motion on an arc centered at a shoulder of the user in the two-dimensional plane, obtains, from the three-dimensional acceleration, a direction of a line segment connecting the wrist and the shoulder, and obtains a bending angle of an elbow of the user based on the line segment.

### REFERENCE TO RELATED APPLICATIONS

This application is based upon and claims the benefit of priority from the prior Japanese Patent Application No. 2023-103950, filed on Jun. 26, 2023, the entire contents of which are incorporated herein by reference.

### TECHNICAL FIELD

The present disclosure relates to an electronic device, an arm swing analysis method, and a storage medium.

### DESCRIPTION OF RELATED ART

There is a technique of, with an electronic device that is worn on the human body, such as a wrist (arm) or a waist, measuring and analyzing the exercise state of its wearer, thereby assisting him/her in appropriately keeping or improving exercise. While a person is doing exercise, which includes walking and running (collectively referred to as “running/walking”), various cyclic changes due to his/her arm swing and so forth occur in acceleration and angular velocity. Measurement results of these cyclic changes can be used to evaluate the user's posture and its' change, deviation, inclination and/or the like during running/walking, in addition to the user's running/walking amount or amount per unit time.

One of such evaluation values is an elbow bending angle (elbow angle), which is disclosed, for example, in JP 2016-146987 A. This elbow angle may be needed to calculate other evaluation values. Conventionally, the angle is obtained by using a plurality of sensors.

### SUMMARY OF THE INVENTION

According to an aspect of the present disclosure, there is provided an electronic device including at least one processor that identifies a cyclic change related to an arm swing of a user from a three-dimensional acceleration measured by a measurer,
wherein the at least one processor
   defines a two-dimensional plane containing a plane of the arm swing of the user from the three-dimensional acceleration measured at a wrist of the user,
   detects the cyclic change as a motion on an arc centered at a shoulder of the user in the two-dimensional plane,
   obtains, from the three-dimensional acceleration, a direction of a line segment connecting the wrist and the shoulder, and
   obtains a bending angle of an elbow of the user based on the line segment.

### BRIEF DESCRIPTION OF DRAWINGS

The accompanying drawings are not intended as a definition of the limits of the invention but illustrate embodiments of the invention, and together with the general description given above and the detailed description of the embodiments given below, serve to explain the principles of the invention, wherein:

FIG. 1 shows an overall configuration of a measurement system of a first embodiment;

FIG. 2 shows a worn state of an electronic device, where the electronic device is worn on an arm of a user;

FIG. 3 is a block diagram showing a functional configuration of the electronic device;

FIG. 4A illustrates measurement by an acceleration sensor;

FIG. 4B illustrates acceleration components during arm swing;

FIG. 5 illustrates measurement of the arm swing and calculation of the angle of the arm swing;

FIG. 6 is a flowchart showing a control procedure of an index calculation control process in the first embodiment;

FIG. 7 is a flowchart showing a control procedure of an arm swing acceleration calculation process in the first embodiment;

FIG. 8 illustrates how to obtain the angle of the arm swing of a modification;

FIG. 9 is a flowchart showing a control procedure of the index calculation control process in the modification;

FIG. 10A illustrates a worn state of an electronic device of a second embodiment;

FIG. 10B is a block diagram showing a functional configuration of the electronic device of the second embodiment;

FIG. 11 illustrate an acceleration related to the arm swing in the second embodiment;

FIG. 12 is a flowchart showing a control procedure of a measurement control process in the second embodiment;

FIG. 13 is a flowchart showing a control procedure of the index calculation control process in the second embodiment; and

FIG. 14 is a flowchart showing a control procedure of the arm swing acceleration calculation process in the second embodiment.

### DETAILED DESCRIPTION

Hereinafter one or more embodiments will be described with reference to the drawings.

### First Embodiment

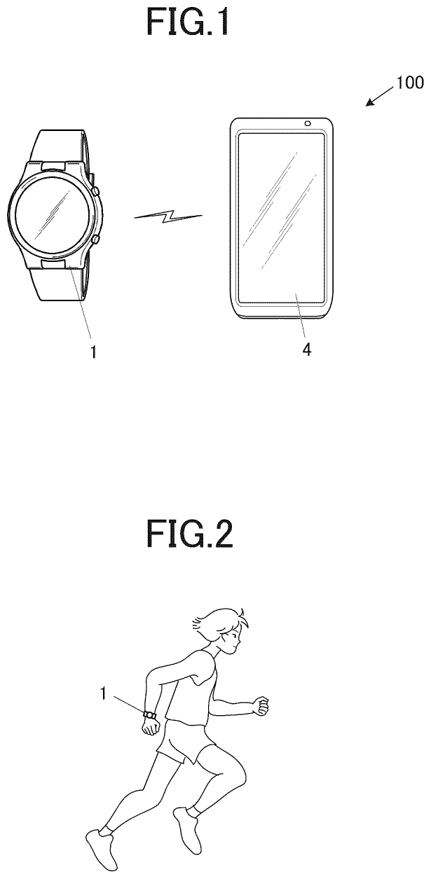

FIG. 1 shows an overall configuration of a measurement system 100 of a first embodiment.

The measurement system 100 includes an electronic device 1 and an electronic device 4.

The electronic device 1 is an arm-worn device, such as an electronic wristwatch or a smartwatch. The electronic device 1 is worn on an arm, a wrist in particular, of a user, to mainly measure the motion state of the wrist during activities, such as walking and running.

FIG. 2 shows the electronic device 1 worn on an arm.

The electronic device 4 is a terminal device, such as a smartphone, but may be a general-purpose electronic computer. The electronic device 1 and the electronic device 4 can communicate with one another by wireless communications, such as Bluetooth® communications or wireless LAN communications. The electronic device 1 and the electronic device 4 may not be connected with one another at all times during an activity. Hereinafter the electronic device 1 will be described as being communicatively connected with the electronic device 4 during an activity, though.

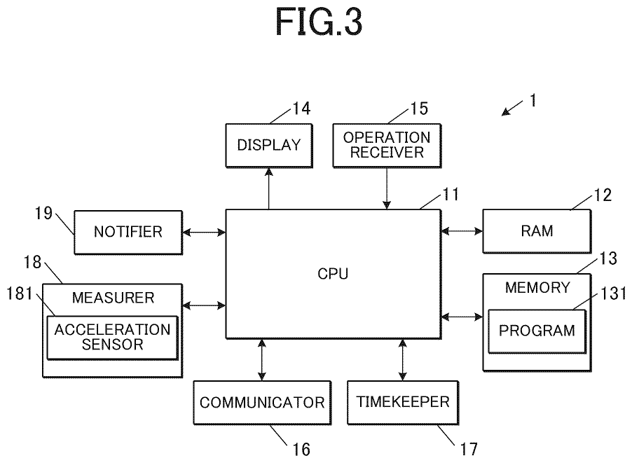

FIG. 3 is a block diagram showing a functional configuration of the electronic device 1.

The electronic device 1, such as an electronic wristwatch, includes a central processing unit (CPU) 11, a random access memory (RAM) 12, a memory 13, a display 14, an operation receiver 15, a communicator 16, a timekeeper 17, a measurer 18 (first measurer), and a notifier 19.

The CPU 11 is a processor that performs arithmetic processing. The CPU 11 performs arithmetic processing and overall control of operation (actions) of the electronic device 1. The CPU 11 may have a single processor or two or more processors that perform arithmetic processing in parallel or operate independently depending on the usage. The CPU 11 corresponds to a computer of the present disclosure.

The RAM 12 provides the CPU 11 with a memory space for working. The RAM 12 stores temporary data.

The memory 13 is, for example, a nonvolatile memory, such as a flash memory. The memory 13 stores a program(s) 131, setting data and so forth. The program 131 controls execution of data processing and analysis on data measured during an activity, which will be described later. In particular, the program 131 controls a process of calculating an arm swing index(es) by identifying a cyclic change(s) related to the arm swing in walking or running (running/walking) of a user.

The display 14 displays various types of information under the control of the CPU 11. The display 14 has a display screen, such as a liquid crystal display or an organic electro-luminescent (EL) display. The display 14 may be capable of color display or display in a limited number of colors.

The operation receiver 15 includes a pushbutton switch(es) to receive input operations from the outside. The operation receiver 15 may include a touchscreen in addition to or instead of the pushbutton switch. The touchscreen is superimposed on the display screen.

The communicator 16 controls communications with external devices. The communicator 16 can control communications in accordance with communication protocols of Bluetooth, wireless LAN or the like as described above. The communicator 16 may include a module for Bluetooth communications. Alternatively, the communicator 16 may include a network card for wireless LAN communications.

The timekeeper 17 measures the passage of time on the basis of clock signals of a certain frequency generated by a not-shown oscillation circuit and keeps the current time. The timekeeper 17 may include, for example, a real time clock (RTC). Further, the timekeeper 17 may include a theoretical regulation circuit that adjusts a gap between the oscillation frequency of the oscillation circuit and the passage of time. The timekeeper 17 may not include a dedicated counter for measuring the passage of time, and the CPU 11 may count outputs of the theoretical regulation circuit.

The measurer 18 includes an acceleration sensor 181. The acceleration sensor 181 measures accelerations in directions of three axes orthogonal to one another (three-dimensional acceleration).

The notifier 19 performs a predetermined notifying action(s), other than the display 14 performing display on the display screen. Examples of the notifying action include generation of beeps, output of sounds, output of vibrations, and light emission or light blinking by a light emitting element. The notifier 19 may include a well-known component(s) for some or all of these actions.

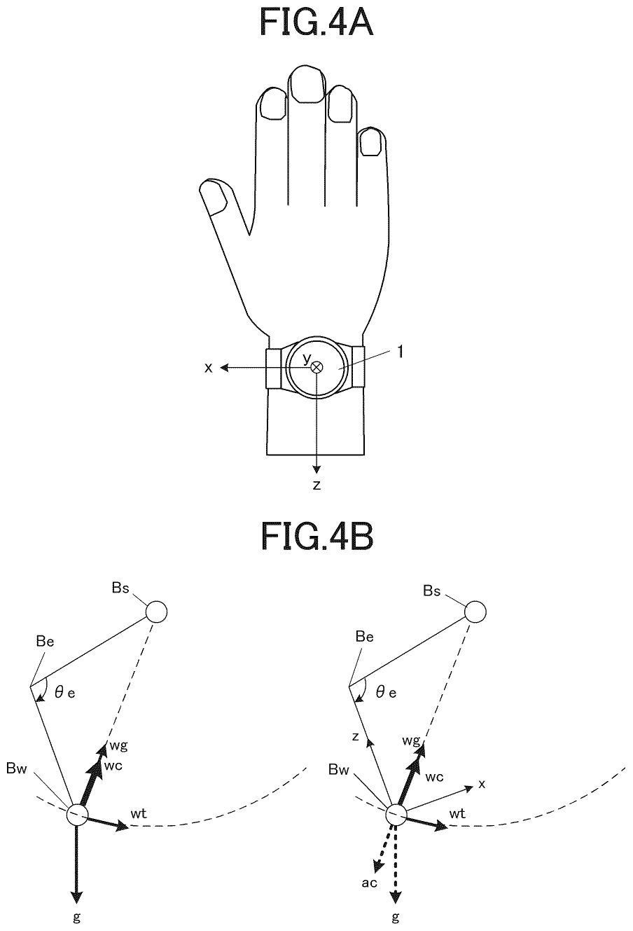

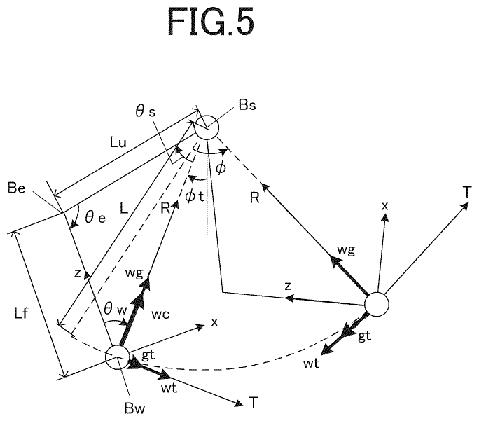

FIG. 4A and FIG. 4B illustrate measurement by the acceleration sensor 181.

The acceleration sensor 181 measures the three-dimensional acceleration in a coordinate system represented by an x-axis and a z-axis orthogonal to one another along the display surface of the electronic device 1 fixed to a wrist of its user and a y-axis perpendicular to the display surface inward as shown in FIG. 4A. With the electronic device 1 worn on the wrist, the z-axis is usually the extension direction of the arm. In other words, the z-axis direction is the direction from the wrist toward the elbow of the user. The x-axis direction is orthogonal to the y-axis and the z-axis, is substantially parallel to the back of the hand, and is the direction from the little finger toward the thumb. The arm swing usually occurs in the xz plane, which is a two-dimensional plane, except when a hand turning/flipping motion, which will be described later, is performed.

As shown in FIG. 4B, if the user who wears the electronic device 1 runs or walks swinging his/her arms, his/her arms each extend from the shoulder Bs to the wrist Bw bending at the elbow Be. The wrist Bw moves back and forth, namely, performs one reciprocating motion in every two steps, along a pendulum-like path with the shoulder Bs as a supporting position.

At the time, in a coordinate system at rest shown on the left side in FIG. 4B, force received by the wrist Bw is resultant force of gravity (gravitational acceleration g) and force received from the arm. The force received from the arm is the sum of stress (acceleration wg) against the gravity, centripetal force (acceleration WC, i.e., centripetal acceleration) generated according to the pendular motion, and force (acceleration wt) received from the arm swing motion with muscular force or the like of the arm. In the middle of the arm swing, the stress against the gravity does not necessarily offset, of the gravity, a component along the movement direction of the arm. That is, the stress only offsets, of the gravity, a component in the direction connecting the shoulder Bs and the wrist Bw.

If this situation is viewed in the coordinate system (in the xz plane) fixed to the wrist Bw, it turns to be a rotating coordinate system shown on the right side in FIG. 4B. The wrist Bw receives centrifugal force (acceleration ac) and also receives, from the arm, stress (acceleration wc) that offsets the centrifugal force ac.

The acceleration sensor 181 is configured such that an acceleration detection element is supported by a support member and detects force received from the support member. Therefore, the gravity and the centrifugal force received by the detection element and the support member simultaneously are not detected by the acceleration sensor 181, which are indicated by dotted lines in FIG. 4B. At the wrist Bw, the acceleration sensor 181 of the electronic device 1 detects the sum of the accelerations wc, wg, wt corresponding to stress.

Next, measurement of the arm swing will be described.

FIG. 5 illustrates measurement of the arm swing and calculation of the angle of the arm swing. In FIG. 5, the position of the shoulder Bs is shown as being fixed. That is, FIG. 5 shows positional change according to the arm swing only.

As described above, in the duration of one cycle (two steps) of the arm swing, the elbow bending angle (elbow angle θe) hardly changes. Therefore, the wrist Bw can be considered to perform the reciprocating motion on an arc having a radius L and centered at the shoulder Bs. That is, the radius L is the distance between the shoulder Bs and the wrist Bw. In the paper surface (arm swing plane) of FIG. 5, the angle formed by two line segments connecting the shoulder Bs and the wrist Bw at both ends (turn points) where the direction of the arm swing changes (switches) is the angle (magnitude) of the arm swing.

A two-dimensional plane is defined by the wrist Bw as the origin, the direction therefrom to the shoulder Bs as an R-axis direction, and the direction that is perpendicular to the R-axis direction and is the movement direction of the wrist Bw as a T-axis direction. In this embodiment, the plane containing the R-axis and the T-axis is parallel (identical) to the plane containing the x-axis and the z-axis. A coordinate system represented by the R-axis and the T-axis also rotates according to the arm swing. The angular difference θw (first angle) between the R-axis and z-axis or the T-axis and x-axis depends on the elbow angle de, which is the elbow bending angle, and remains the same under the abovementioned condition that the elbow angle θe is fixed. In this two-dimensional coordinate system fixed to the wrist, in the R-axis direction (direction perpendicular to the arc on/along which the wrist Bw reciprocates), the accelerations wg, wc corresponding to stress are generated, and an acceleration in the T-axis direction is the sum of the acceleration wt corresponding to the arm swing and an acceleration gt of the T-axial component of the gravitational acceleration g.

Of these acceleration components, the acceleration wt in the T-axis direction fluctuates sinusoidally to the positive and the negative centered at wt=0 in each cycle of the arm swing. In the normal arm swing, the position of the wrist Bw is usually lower than the position of the shoulder Bs in the vertical direction. Therefore, the accelerations wg, wc in the R-axis direction each contain a positive offset value (direct-current (DC) component) with respect to the sinusoidal periodic (cyclic) oscillation. Performing low-pass filtering (LPF) on change in acceleration in the plane containing the R-axis and the T-axis (i.e., the x-axis and the z-axis) selectively extracts the offset component (sum of the offset component of the acceleration wg and the offset component of the acceleration wc), which is the DC component, in the R-axis direction of the acceleration. This acceleration direction in the xz plane of the coordinate system fixed to the wrist is identified as the R-axis direction. That is, once a vector (xr, zr) indicating the R-axis direction is determined, the angular difference θw from the z-axis to the R-axis is obtained by, for example, an inner product of this vector and the z-axis direction (0, 1). Once the R-axis direction is identified, the direction of the T-axis, which is orthogonal to the R-axis, is also identified. In the R-axis direction actually obtained, namely, the angular difference θw, some variation exists. Therefore, for example, a representative value, such as the average value or the median value, of angular differences θw(t) obtained at respective timings t in one cycle of the arm swing may be determined as the angular difference θw in this cycle. The LPF may be performed by, for example, after setting a moving window appropriate for the cutoff frequency and performing FFT on data in the moving window, cutting the spectral intensity of a frequency (ies) equal to or higher than the cutoff frequency and performing inverse transformation. Alternatively, more simply, the average value in the moving window may be subtracted. Still alternatively, more step-by-step, the most recent angular difference θw(t) multiplied by a coefficient k for the number of data in a reference period of the LPF and an addition average value up to the previous time multiplied by a coefficient (1−k) may be added up to obtain a new addition average value, and the obtained addition average value may be subtracted from the angular difference θw(t). Measured data that are unneeded because they are outside the range of measured data to be used in the LPF may be deleted in order.

As mentioned above, since the gravitational acceleration g is not measured, the acceleration gt of its T-axial component is not included in the measured values by the acceleration sensor 181. However, the sum of the acceleration wg of the R-axial component and the acceleration gt of the T-axial component is the gravitational acceleration g. Therefore, once the acceleration wg is identified, the acceleration gt of the T-axial component can be calculated. That is, the acceleration component in the direction along the T-axis is identified as the sum of the acceleration wt and the acceleration gt.

The stress (acceleration wc) corresponding to the centrifugal force depends on the movement velocity v of the wrist Bw, namely, wc=v²/L. At both ends of the arm swing (turn points of the arm swing), the movement velocity v=0. The movement velocity v of the wrist Bw is obtained by integrating the sum of the acceleration wt of the arm swing and the acceleration gt of the T-axial component of the gravitational acceleration from a turn point. The T-axial component of the acceleration to be measured is substantially equal to the acceleration wt. Therefore, the movement velocity v at each timing is obtained, after the acceleration gt of the T-axial component is calculated as described above, by accumulating (integrating) the acceleration wt+gt using a turn point as a reference. Because the accumulation is reset at each turn point, accumulation of errors is minimized.

The turn points are each obtained, for example, by the following procedure.

On the basis of the R-axis direction and the T-axis direction identified as described above, the acceleration wt in the T-axis direction, namely, the arm swing direction, is obtained. By integrating the acceleration wt from a certain point of time as a reference, velocities vt in the arm swing direction at respective points of time are tentatively calculated. Since the velocity at the certain point of time as the reference (initial velocity) is not identified, the velocities vt are each a value with an offset. The value (average value) obtained by averaging the obtained velocities vt at the respective points of times is the value of the offset. In the situation where no turn point is identified, the phase of the arm swing cannot be identified. Hence, a period used for performing the above averaging needs to be sufficiently longer than one cycle of the arm swing. For example, the period used for performing the averaging may be around eight to ten seconds. By subtracting the obtained average value (the value of the offset) from each velocity vt obtained earlier, the movement velocity v is determined accurately. The points at which the velocities vt are zero are the turn points.

Before the first turn point is identified (before the R-axis direction and the T-axis direction are identified) or when the arm swing is too small to be distinct, it is difficult to identify, by the above-described integration, the points at which the movement velocity v is zero. In such a case, the turn point(s) of the arm swing may be estimated by another method, for example, from the timing at which the acceleration in the x-axis direction becomes the maximum (or minimum) value. Time between adjacent turn points is time required for swing an arm once (i.e., moving an arm either forward or backward), and time required for swinging an arm twice, namely, swinging an arm and returning the arm to its original position or near there, is one cycle of the arm swing, which hereinafter may be referred to as “arm swing cycle”.

The value obtained by subtracting the acceleration wc corresponding to the movement velocity v from an acceleration wr, which is the R-axial component of the measured acceleration, is the acceleration wg (i.e., wr=wc+wg). The angle φt formed by the R-axis direction at a certain timing t and the vertical direction is obtained as follows: φt=cos⁻¹ (wg/g). Therefore, the acceleration gt of the T-axial component of the gravitational acceleration g is obtained as follows: gt=g·sin (φt).

The radius L is determined by an upper arm length Lu, a forearm length Lf and the elbow angle θe. The arm length Lu+Lf statistically depends on the height. The ratio of the length Lu to the length Lf is basically a fixed value. The ratio of the height to the arm length Lu+Lf and the ratio of the length Lu to the length Lf are predetermined and stored in the memory 13. By the electronic device 1 obtaining the user's height information in advance, once the elbow angle θe is determined, the radius L is obtained. The values of these ratios may not be fixed. They may be set more finely on the basis of information such as the age, gender, race (country), weight, and/or foot size and held. The ratio of the length Lu to the length Lf may be 1 to 1. Alternatively, the lengths Lu, Lf may be determined by the operation receiver 15 receiving user's direct inputs of measured values.

In the case where the lengths Lu, Lf are different from one another, if a perpendicular line is drawn from the wrist Bw to a line segment Bs-Be (or its extension line), the length L1 of the perpendicular line is expressed as follows: L1=Lf·sin (θe), and the length L2 from the shoulder Bs to an intersection point with the perpendicular line is expressed as follows: L2=Lu-Lf·cos (Ge). Therefore, L=(L1²+L2²)^{1/2}=(Lu²+Lf²−2Lu·Lf·cos (θe))^{1/2}.

In the triangle formed by the shoulder Bs, the elbow Be and the wrist Bw, the elbow angle θe is obtained by subtracting the sum of the angle θs of the shoulder Bs and the angle of the wrist Bw (angular difference θw) from 180. If the lengths Lu, Lf are determined in addition to the angular difference θw obtained as described above, the angle θs is identified. For example, on the basis of the length of a perpendicular line drawn from the elbow Be to the line segment Bs-Bw, Lf·sin (θw)=Lu·sin (θs). Therefore, θs=sin⁻¹ ((Lf/Lu) sin (θw)).

In the case where the ratio of the length Lu to the length Lf is 1 to 1, the triangle formed by the shoulder Bs, the elbow Be and the wrist Bw is an isosceles triangle. That is, the angular difference θw is equal to the angle θs. In this case, L=Lu·(2(1−cos (θe)))^{1/2}, and the elbow angle θe=180−2·θw. Therefore, the radius L may be obtained as follows: L=2·Lu·cos (θw).

By further integrating the movement velocity v (i.e., integrating the acceleration component in the T-axis direction twice; accumulating or adding up in numerical processing of discrete values) from the turn point, the movement amount (movement distance) Lt of the arm from the turn point is obtained. The movement amount Lt is the length of the arc. Therefore, the angle φ corresponding to the movement amount Lt is calculated as follows: φ[rad]=Lt/L. The angle φ between turn points is obtained as an arm swing angle φ0 (magnitude of the arm swing) in a half cycle. Since there may be variation in measurement and/or arm swing itself, arm swing angles θ0 obtained multiple times may be averaged and output. These arm swing cycle and magnitude of the arm swing are included in the arm swing indices that are used for evaluating the arm swing state of the user. The arm swing indices may include the angles of the arm swing at both ends, which will be described later, and the elbow angle θe.

In the above, it is assumed that the user ideally swings his/her arms in the xz plane, but there is a user who rotates his/her wrist(s) on the z axis (turns or flips his/her hand(s)) or moves his/her wrist(s) in the y-axis direction (even when not rotating the wrist(s) on the z-axis) in each cycle of the arm swing while he/she is running/walking. In such a case, an acceleration component that intrinsically appears in the x-axial component may partly or wholly appear in the y-axial component. In this embodiment, a coordinate transformation is performed on the three-axial acceleration (three-dimensional acceleration) measured by the acceleration sensor 181, which is worn on the wrist Bw, to rotate the z-axis such that the acceleration in the y-axis direction becomes zero. This coordinate transformation puts the three-dimensional acceleration on the xz plane to calculate the arm swing angle. The magnitude of the x-axial component after the transformation is expressed as follows: sgn (x)·(x²+y²)^{1/2}. If it can be assumed that the force in the y-axis direction is not applied to the arm swing of the user at all, or the force in the y-axis direction is negligibly small, the above coordinate transformation may not be performed.

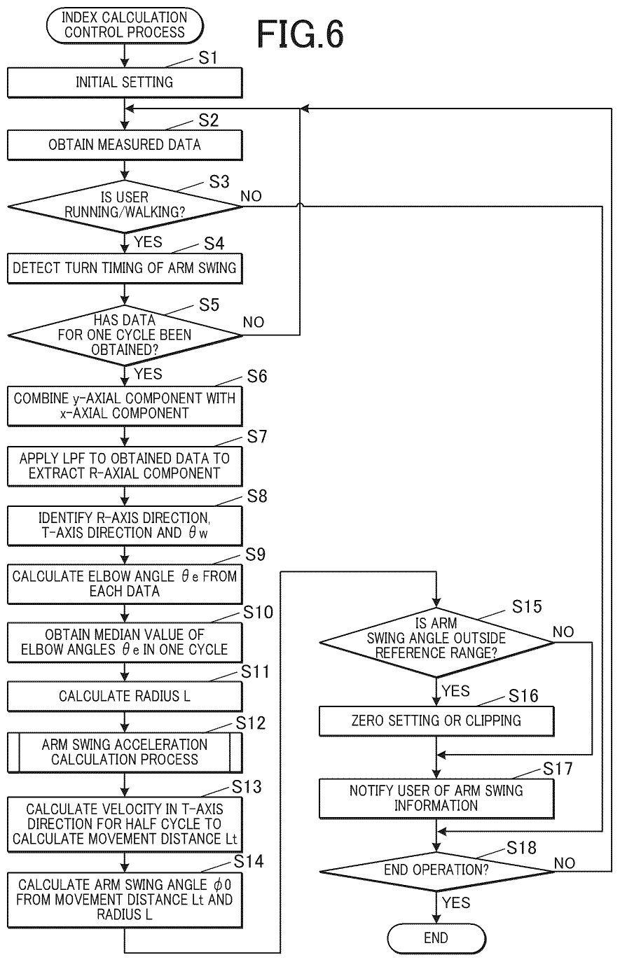

FIG. 6 is a flowchart showing a control procedure of an arm swing index calculation control process that is performed by the CPU 11 of the electronic device 1 in this embodiment. This control process, which includes an arm swing analysis method in this embodiment, is started, for example, with a start command for activity amount measurement from an application/software program for a walking- or running-related activity, and is continuously and repeatedly performed until an end command for the measurement is received.

The CPU 11 performs initial setting (S1). The CPU 11 sets the upper arm length Lu and the forearm length Lf of the user from the value of his/her height obtained. As described above, the CPU 11 may determine the lengths Lu, Lf further on the basis of the information such as the age, gender and/or the like of the user, or may set, as the lengths Lu, Lf, user's direct inputs as they are.

The CPU 11 obtains measured data at set intervals from the acceleration sensor 181 (S2). The set interval(s) is an interval corresponding to a predetermined temporal resolution. Each measured data is expressed in the above-described xyz coordinate system.

The CPU 11 determines, using data of the most recent set (point of) time among the measured data, whether the user is running/walking (S3). During running/walking, the cyclic fluctuation related to the arm swing appears in the values measured by the acceleration sensor 181 in every one step and/or two steps, as described above. On the measured values, not only the cyclic fluctuation related to the arm swing but also cyclic fluctuation of a specific directional component due to, for example, an up-and-down motion corresponding to legs moving up and down (off the ground and on the ground) may be superposed. The CPU 11 may determine that the user is running/walking if, in the range of cycles (frequencies) assumed as running/walking, vibrations having an amplitude equal to or greater than a reference value continue for a reference time or more. The CPU 11 may distinguish running and walking from one another. Further, depending on the type of an activity being set, the CPU 11 may determine that the user is not running/walking if he/she is outside the set state (e.g., walking in the case where running is set, or running in the case where walking is set).

If the CPU 11 determines that the user is not running/walking (S3; NO), the process of the CPU 11 proceeds to S18. If the CPU 11 determines that the user is running/walking (S3; YES), the CPU 11 detects a turn timing of the arm swing (S4). As described above, the turn timing is the timing at which the movement velocity v is zero and before and after which the movement velocity v switches between the positive (plus) and the negative (minus). If the movement velocity v is difficult to be identified, the turn timing is estimated by another method, for example, from change in the acceleration in the x-axis direction. The CPU 11 may detect the turn timing by any other suitable method.

The CPU 11 determines whether data for one cycle of the arm swing has been obtained (S5). One cycle of the arm swing is from the last turn timing of the arm swing of the previous cycle to the second turn timing after that. If the CPU 11 determines that data for one cycle of the arm swing has not been obtained (S5; NO), the process of the CPU 11 returns to S2.

If the CPU 11 determines that data for one cycle of the arm swing has been obtained (S5; YES), the CPU 11 performs a coordinate transformation on, of each data included in the data for one cycle of the arm swing, three-dimensional data (data in directions of three axes) to combine the y-axial component with the x-axial component (S6).

The CPU 11 applies a low-pass filter to (i.e., performs low-pass filtering on) the data for one cycle (or data for a plurality of cycles including the previous cycle(s)) of the arm swing to extract the steady R-axial component, namely, the gravitational acceleration component, remaining at each timing (S7). The CPU 11 identifies, about each data included in the data for one cycle of the arm swing, the R-axis direction, the T-axis direction orthogonal to the R-axis direction, and the angular difference θw (S8).

The CPU 11 calculates, from each data included in the data for one cycle of the arm swing, the elbow angle de, using the initially set lengths Lu, Lf and the angular difference θw (S9). The processes in S8, S9 and so forth constitute the identifying means of this embodiment. The CPU 11 obtains the median value of the elbow angles φe calculated from the respective data included in the data for one cycle of the arm swing as the representative value of the elbow angle θe (S10). The representative value may be the average value as described above. The CPU 11 calculates the radius L using the lengths Lu, Lf and the elbow angle θe (S11). As described above, if the lengths Lu, Lf are equal, the CPU 11 may calculate the radius L using the angular difference θw instead of the elbow angle θe.

The CPU 11 performs an arm swing acceleration calculation process (S12), which will be detailed later. In the arm swing acceleration calculation process, the CPU 11 transforms the three-axial acceleration (three-dimensional acceleration) measured at each timing into data in the RT coordinate system. The CPU 11 calculates the acceleration gt of the T-axial component of the gravitational acceleration and the movement velocity v about each data in order.

The CPU 11 integrates the movement velocity v for each half cycle to calculate the movement amount Lt along the arc (S13). The CPU 11 calculates the arm swing angle φ0 for each half cycle from the movement amount Lt and the radius L (S14). The CPU 11 may take the average value of the two arm swing angles φ0 as the arm swing angle in the cycle concerned. Alternatively, the CPU 11 may calculate, in S13, the average value of the two movement amounts Lt, and obtain, on the basis of this average value, the arm swing angle in the cycle concerned.

The CPU 11 determines whether the obtained arm swing angle is outside a reference range (S15). If the CPU 11 determines that the arm swing angle is outside the reference range (S15; YES), the CPU 11 adjusts the arm swing angle (S16). If the arm swing angle is greater than the upper limit value of the reference range, the CPU 11 clips the arm swing angle at the upper limit value of the reference range (S16). The arm swing angle being too large is unmeaningful, and therefore output of up to the meaningful maximum value are enough. If the arm swing angle is smaller than the lower limit value of the reference range, the CPU 11 sets the arm swing angle to zero. When the arm swing angle is too small, the acceleration is also small, so that, usually, the arm swing angle cannot be calculated accurately. Therefore, if the arm swing angle obtained is smaller than a reference (i.e., lower limit value of the reference range), the CPU 11 may perform output as the arm swing not being performed. Alternatively, in such a case, the CPU 11 may output the arm swing angle as an error. That is, the CPU 11 may simply indicate that the arm swing has not been detected accurately. After S16, the process of the CPU 11 proceeds to S17. If the CPU 11 determines that the arm swing angle is not outside the reference range (S15; NO), the process of the CPU 11 proceeds to S17.

In S17, the CPU 11 notifies the user of the obtained arm swing information (S17). More specifically, the CPU 11 may cause the display 14 to graphically display the history (records) of the arm swing angle, for example. The CPU 11 may determine whether the arm swing angle is in an appropriate range, and output the determination result. This output may be output of sounds by the notifier 19 in addition to or instead of output (display) by the display 14. The output by the notifier 19 may be performed with a specific vibration pattern or light emission pattern.

The CPU 11 determines whether the operation receiver 15 has received an end operation to end the activity measurement (S18). If the CPU 11 determines that the operation receiver 15 has not received the end operation (S18; NO), the process of the CPU 11 returns to S2. If the CPU 11 determines that the operation receiver 15 has received the end operation (S18; YES), the CPU 11 ends the index calculation control process. Before ending the index calculation control process, the CPU 11 may write data in the RAM 12 necessary to save into the memory 13.

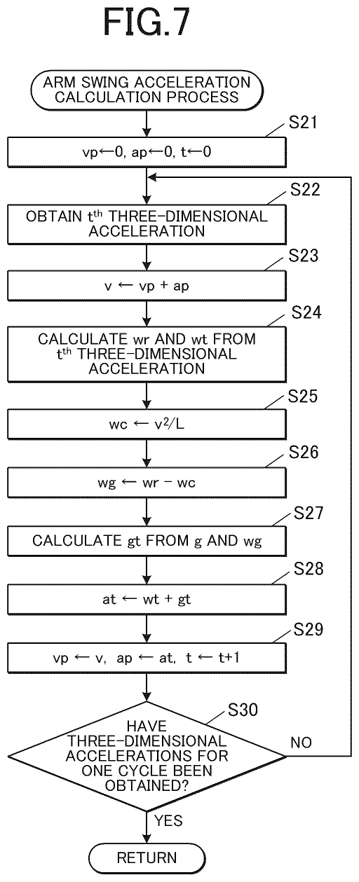

FIG. 7 is a flowchart showing a control procedure of the arm swing acceleration calculation process that is performed by the CPU 11 in S12.

The CPU 11 assigns, as initial values, “0” to variables vp, ap, t (S21).

The CPU 11 obtains the t^{th} data from the top among the plurality of three-axial acceleration data (three-dimensional acceleration data) for one cycle of the arm swing (S22), which the CPU 11 has determined in S5 as having been obtained. The CPU 11 assigns the sum of the variables vp, ap to the movement velocity v (S23).

The CPU 11 performs a coordinate transformation on the obtained t^{th} three-axial acceleration data, and calculates the acceleration wr of the R-axial component and the acceleration wt of the T-axial component on the basis of the angular difference θw identified in S8 (S24). The CPU 11 calculates the acceleration wc, which corresponds to the stress against the centrifugal force, from the movement velocity v and the radius L (S25).

The CPU 11 subtracts the acceleration wc from the acceleration wr to calculate the acceleration wg of the R-axial component of the gravitational acceleration g (S26). The CPU 11 calculates the acceleration gt of the T-axial component of the gravitational acceleration g from the gravitational acceleration g and the acceleration wg, which are steady values (S27). The CPU 11 adds up the acceleration wt and the acceleration gt to obtain an acceleration at in the T-axis direction, namely, the arm swing direction (S28).

The CPU 11 assigns the movement velocity v to the variable vp, assigns the acceleration at in the arm swing direction to the variable ap, and adds “1” to the variable t (S29). The CPU 11 determines whether all the three-axial acceleration data for one cycle of the arm swing have been obtained (and processed) (S30). If the CPU 11 determines that all the three-axial acceleration data for one cycle of the arm swing have not been obtained (S30; NO), the process of the CPU 11 returns to S22. If the CPU 11 determines that all the three-axial acceleration data for one cycle of the arm swing have been obtained (S30; YES), the CPU 11 ends the arm swing acceleration calculation process and returns its process to the index calculation control process.

### [Modification]

Instead of being obtained by integrating twice (accumulating) the acceleration at in the arm swing direction of the wrist Bw, the arm swing width may be obtained from the difference between the positions (angles) of the wrist Bw at the turn points that are both ends of the arm swing. The positions of the turn points may be expressed by angles with the vertically downward direction as a reference.

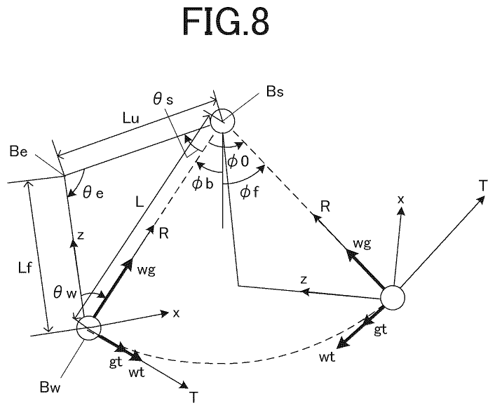

FIG. 8 illustrates how to obtain the arm swing angle φ0 in a modification.

For example, as described above, the angle φt at a certain timing t is obtained as follows: φt=cos⁻¹ (wg/g). Since the movement velocity v of the arm swing is zero at each turn point, the acceleration wc corresponding to the centrifugal force is also zero thereat. Hence, wg=wr. Therefore, the angle φt is easily obtained by using the acceleration wr that is, of the gravitational acceleration, the measured value of the component perpendicular to the arc, namely, as follows: φt=cos⁻¹ (wr/g).

The arm swing angle φ0 is obtained by summing the angle of (first turn point angle), which is the angle φt when the wrist Bw is at the most forward position, and the angle φb (second turn point angle), which is the angle φt when the wrist Bw is at the most backward position. If the wrist Bw does not move backward from the vertically downward position, the angle φb is negative.

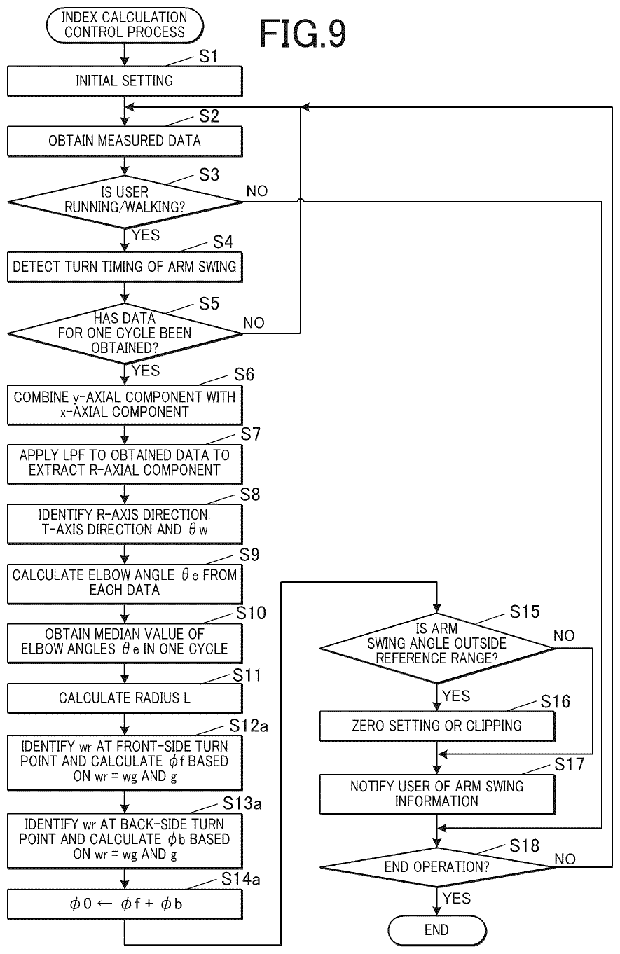

FIG. 9 is a flowchart showing a control procedure of the index calculation control process, which is performed by the CPU 11, in the modification. The index calculation control process of this modification is the same as that of the first embodiment except that S12-S14 have been replaced by S12a-S14a. Other than the above, there is no difference. The same process contents are provided with the same reference signs, and detailed descriptions thereof are omitted.

After calculating the radius L in S11, the CPU 11 identifies, in accordance with the R-axis direction and the T-axis direction at the front-side turn point, the acceleration wr at this turn timing (S12a). The CPU 11 calculates the angle of on the basis of the acceleration wr and the gravitational acceleration g (S12a).

The CPU 11 identifies, in accordance with the R-axis direction and the T-axis direction at the back-side turn point, the acceleration wr at this turn timing (S13a). The CPU 11 calculates the angle φb on the basis of the acceleration wr and the gravitational acceleration g (S13a).

The CPU 11 adds up the angles φf, φb, thereby calculating the arm swing angle φ0 (S14a). Then, the process of the CPU 11 proceeds to S15. The angles φf, φb may be included in the indices that are used for evaluating the arm swing. Therefore, the CPU 11 may store and hold, together with the arm swing angle φ0, the angles φf, φb as evaluation information.

### Second Embodiment

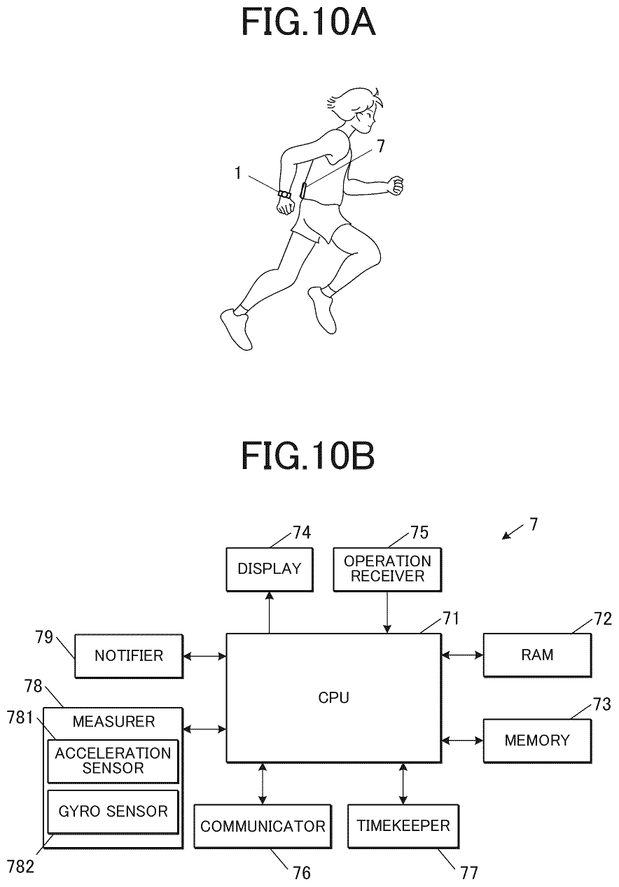

FIG. 10A illustrates the worn state of an electronic device of a second embodiment.

In this embodiment, as shown in FIG. 10A, the user who performs an activity wears an electronic device 7 that is fixed to his/her waist, in addition to the electronic device 1 that is fixed to his/her arm. The electronic device 7 measures the motion state at the position of the waist. The electronic device 7 includes a three-axis gyro sensor 782 in addition to an acceleration sensor 781.

FIG. 10B is a block diagram showing a functional structure of the electronic device 7.

The electronic device 7 includes a CPU 71, a RAM 72, a memory 73, a display 74, an operation receiver 75, a communicator 76, a timekeeper 77, a measurer 78 (second measurer), and a notifier 79.

The CPU 71 is a processor that performs arithmetic processing and controls operation (actions) of the electronic device 7. The CPU 71 may have a single processor or two or more processors that perform arithmetic processing in parallel or operate independently depending on the usage.

The RAM 72 provides the CPU 71 with a memory space for working and stores temporary data. The memory 73 is a nonvolatile memory, such as a flash memory, and stores a program(s), setting data and so forth.

The display 74 displays various types of information and so forth under the control of the CPU 71. If measured data can be basically viewed on an external device, the display screen of the display 74 may be minimal. The display screen is, for example, a liquid crystal display or an organic EL display. The display 74 may be capable of color display or display in a limited number of colors.

The operation receiver 75 includes a pushbutton switch(es) to receive input operations from the outside. The operation receiver 75 may include a touchscreen in addition to or instead of the pushbutton switch. The touchscreen is superimposed on the display screen.

The communicator 76 controls communications with external devices. The communicator 76 can control communications in accordance with communication protocols of Bluetooth, wireless LAN or the like. In this embodiment, the communicator 76 is capable of transmitting and receiving data to and from the communicator 16 of the electronic device 1 in accordance with common protocols. The communicator 76 may include a module for Bluetooth communications. Alternatively, the communicator 76 may include a network card for wireless LAN communications.

The timekeeper 77 measures the passage of time on the basis of clock signals of a certain frequency generated by a not-shown oscillation circuit and keeps the current time. The timekeeper 77 may include, for example, a real time clock (RTC). Further, the timekeeper 77 may include a theoretical regulation circuit that adjusts a gap between the oscillation frequency of the oscillation circuit and the passage of time. The timekeeper 77 may not include a dedicated counter for measuring the passage of time, and the CPU 11 may count outputs of the theoretical regulation circuit.

The measurer 78 includes the acceleration sensor 781 and the gyro sensor 782. The acceleration sensor 781 is fixed to the waist and measures the three-dimensional acceleration. The gyro sensor 782 is fixed to the waist and measures angular velocities in directions of three axes orthogonal to one another (three-dimensional angular velocity).

The notifier 79 performs a predetermined notifying action(s), other than the display 74 performing display on the display screen. Examples of the notifying action include generation of beeps, output of sounds, output of vibrations, and light emission or light blinking by a light emitting element. The light emitting element may be capable of emitting light while switching emission colors of light. The notifier 79 may include a well-known component(s) for some or all of these actions.

Next, measurement of the arm swing angle in the second embodiment will be described.

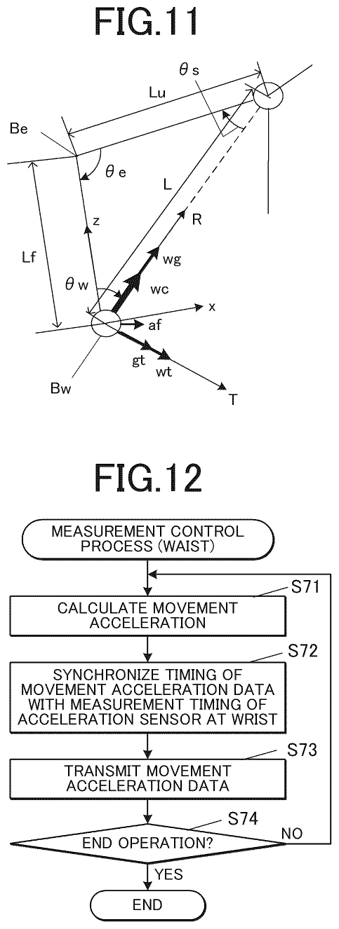

FIG. 11 illustrates the acceleration related to the arm swing in this embodiment.

As described in the first embodiment, the arm swing is expressed by the arc-shaped motion/movement centered at the shoulder Bs. The acceleration related to the arm swing along the arc is given by the sum of the acceleration gt of the T-axial component of the gravitational acceleration g and the acceleration wt corresponding to (received from) the arm swing motion with muscular force or the like of the arm.

In reality, however, since the user is running/walking, on the measured acceleration, an acceleration component (movement acceleration af) corresponding to the movement of the user's body (shoulder Bs) is further superposed. It is difficult to separate this component with the acceleration sensor 181 at the wrist Bw alone.

The measurement system 100 of this embodiment includes the electronic device 7, which is attached to the waist, separately from the electronic device 1, to detect the movement acceleration af related to the user's running/walking. The waist is an example of a suitable position where the acceleration component corresponding to the movement of the user's body is detectable but the acceleration due to the user's arm swing is not detected. The movement acceleration af measured by the electronic device 7 is transmitted to the electronic device 1 and combined with the data measured by the electronic device 1, for example, the movement acceleration af is subtracted from the acceleration of the T-axial component obtained by the electronic device 1, so that the value of the acceleration more closely corresponding to the arm swing is obtained.

Since the arm swing acceleration is the one in the direction along the arc as described above, the accelerations wt, gt as the T-axial components do not match the movement acceleration af depending on the phase of the arm swing, but in this embodiment, the movement acceleration af in the horizontal direction is subtracted as it is, as a fixed value. Even this process improves the accuracy of the measured values related to the arm swing.

The electronic device 7 calculates the movement acceleration af with a well-known method. For example, the electronic device 7 uses a Kalman filter or the like on the measured data by the gyro sensor 782 to identify the vertical direction and identify a movement direction perpendicular to this direction.

The electronic device 1 and the electronic device 7 are usually different in temporal resolution for measurement and measurement timing(s). The electronic device 7 may perform measurement in sync with measurement by the electronic device 1, or the measurement result (measured data) of the electronic device 7 may be adjusted in conformity with the temporal resolution of the measured data of the electronic device 1 and transmitted to the electronic device 1.

The electronic device 7 may obtain information on the measurement timing of the electronic device 1 in advance. Alternatively, the electronic devices 1, 7 may perform measurement by synchronizing a common timing preset in accordance with an accurate date and time obtained by using satellite positioning or the like. Still alternatively, the electronic device 7 may generate data by performing linear interpolation or the like on its measured data to synchronize the measured data with the information on the measurement timing of the electronic device 1.

FIG. 12 is a flowchart showing a control procedure of an activity measurement control process that is performed by the CPU 71 of the electronic device 7. This process is continuously and repeatedly performed once it is started until an end operation is received.

The CPU 71 calculates the movement acceleration af from data for an appropriate period (e.g., for several steps) using a well-known technique as described above (S71). The period is set for the target timing(s) by the moving window. The CPU 71 synchronizes the timing of the data of the obtained movement acceleration af with the measurement timing information of the electronic device 1 (S72).

The CPU 71 causes the communicator 76 to transmit the data of the movement acceleration af to the electronic device 1 (S73). If there is a delay or the like in the transmission timing from the measurement timing, the CPU 71 may attach, to the data to output/transmit (output data), timing information by which the electronic device 1 can recognize the measurement timing of the output data. The electronic device 7 may not attach the timing information to each of all the measured data.

The CPU 71 determines whether an end operation to end the measurement has been received (S74). If the CPU 71 determines that the end operation has not been received (S74; NO), the process of the CPU 71 returns to S71. If the CPU 71 determines that the end operation has been received (S74; YES), the CPU 71 ends the measurement control process.

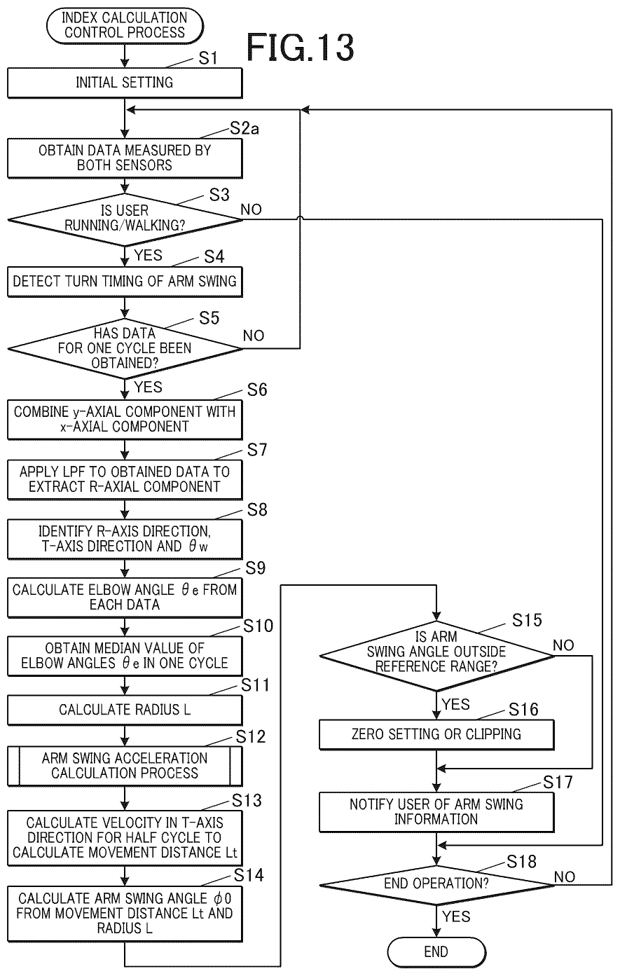

FIG. 13 is a flowchart showing a control procedure of the index calculation control process, which is performed by the CPU 11 of the electronic device 1, in this embodiment. This index calculation control process is the same as that of the first embodiment except that S2 has been replaced by S2a, and the contents of the arm swing acceleration calculation process in S12 have been changed, which will be described below. Other than the above, the index calculation control process of the second embodiment is the same as that of the first embodiment. The same process contents are provided with the same reference signs, and detailed descriptions thereof are omitted.

In each data obtainment loop, the CPU 11 obtains both the measured data by the acceleration sensor 181 and the measured data by the gyro sensor 782 (S2a). The CPU 11 obtains the data from the gyro sensor 782 at a timing corresponding to the data receiving timing of the electronic device 1 (i.e., communicator 16), and accordingly does not necessarily obtain the data at the timing same as that at which the CPU 11 obtains the data from the acceleration sensor 181. After S2a, the process of the CPU 11 proceeds to S3.

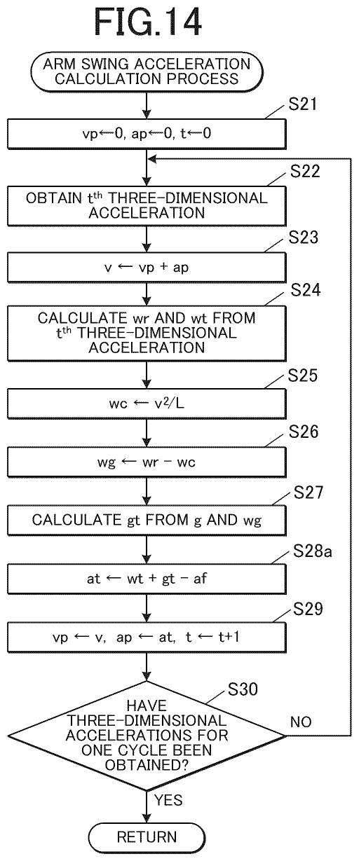

FIG. 14 is a flowchart showing a control procedure of the arm swing acceleration calculation process, which is called in the index calculation control process and performed by the CPU 11, in the second embodiment. This arm swing acceleration calculation process is the same as that of the first embodiment except that S28 has been replaced by S28a. Other than the above, the arm swing acceleration calculation process of the second embodiment is the same as that of the first embodiment. The same process contents are provided with the same reference signs, and detailed descriptions thereof are omitted.

Once the CPU 11 calculates the acceleration gt of the T-axial component of the gravitational acceleration in S27, the CPU 11 subtracts the movement acceleration af from the sum of the accelerations wt, gt, thereby obtaining a value as the acceleration at in the arm swing direction (S28a). Then, the process of the CPU 11 proceeds to S29.

As described above, the electronic device 1 of the above embodiments includes the CPU 11 (at least one processor) that identifies a cyclic change related to the arm swing of a user from the three-dimensional acceleration measured by the measurer 18, which measures the three-dimensional acceleration. The CPU 11 defines a two-dimensional plane (RT plane) containing the plane of the arm swing of the user from the three-dimensional acceleration measured at a wrist (Bw) of the user, detects the cyclic change as a motion on an arc centered at a shoulder (Bs) of the user in the two-dimensional plane, obtains, from the three-dimensional acceleration, the direction of the line segment connecting the wrist Bw and the shoulder Bs, and calculates (obtains) the elbow angle θe based on the line segment.

Conventionally, a user wears a plurality of electronic devices or uses a plurality of measurement devices for his/her arm swing to be evaluated. This increases the cost, weight and processing load of the devices as a whole, and also processes for the measurement and evaluation take much time and effort. To deal with these problems, the present disclosure can provide an electronic device, an arm swing analysis method and a storage medium that can identify the user's elbow bending angle during the arm swing more easily and simply. In the measurement of the acceleration of the wrist Bw, during the arm swing, the degree of inclination of the forearm, namely, the average z-axis direction, is determined according to the elbow angle θe. Meanwhile, since arms basically support wrists against the gravity, the acceleration sensor 181 continuously measures the stress against the gravity. Further, since arms also support wrists against the vertically downward component of the centrifugal force accompanying the arm swing, the acceleration sensor 181 also detects the stress against the centrifugal force cyclically. By identifying the orientations of these components, the electronic device 1 can easily obtain the relationship between the z-axis direction, which is the average orientation of the arm, and the R-axis direction, which is the direction from the wrist Bw to the shoulder Bs, and, by extension, obtains the elbow angle θe. In particular, the electronic device 1 can easily obtain the elbow angle θe from the value(s) measured by the acceleration sensor 181 only. Thus, the electronic device 1 can identify the elbow angle de during the user's arm swing more easily. Therefore, the electronic device 1 can evaluate the user's form, posture and so forth in walking or running more accurately.

Further, the cyclic change (motion) may be a reciprocating motion. Although a person does not necessarily swing his/her arms precisely or monotonously, treating the arm swing as a simple reciprocating motion makes the processes simpler.

Further, the CPU 11 may identify an acceleration in the two-dimensional plane (RT plane) based on the three-dimensional acceleration measured during the arm swing. The CPU 11 may extract the DC component from the acceleration in the two-dimensional plane to identify the direction of the line segment connecting the shoulder Bs and the wrist Bw. The CPU 11 may calculate (obtain) the angular difference θw formed by this line segment and the direction from the wrist Bw to the elbow Be of the user (i.e., the z-axis of the acceleration sensor 181). The CPU 11 may calculate (obtain) the elbow angle θe based on the calculated angular difference θw. In contrast to the vertical component, the acceleration in the back-and-forth direction due to the arm swing is reversely changed cyclically (every half cycle), so that the acceleration in the forward direction and the backward direction are offset over a span of one or more cycles. Therefore, by extracting the DC component as average data by low-pass filtering or the like over this span, it is possible to easily identify the R-axis direction, namely, the direction from the wrist Bw to the shoulder Bs. If the arm length is specified, the elbow angle We can be identified from the angular difference θw. By such a procedure, the electronic device 1 can easily obtain the elbow angle from the measured data of the three-axial acceleration (three-dimensional acceleration).

In particular, the CPU 11 may calculate (obtain) the elbow angle θe based on the upper arm length Lu and the forearm length Lf of the user and the angular difference θw. Thus, if the arm lengths Lu, Lf are obtained, the elbow angle θe can be obtained from the angular difference θw easily. The arm length can be easily measured by the user himself/herself. Therefore, the electronic device 1 can easily obtain the elbow angle θe.

Further, the CPU 11 may calculate (obtain) the elbow angle from each three-dimensional acceleration measured in a period of one or more cycles of the arm swing, thereby obtaining a plurality of elbow angles, and determine the representative value of the elbow angle from the obtained plurality of elbow angles. Although the elbow angle θe often remains substantially unchanged in the range of several cycles of the arm swing, it may differ locally depending on the user's habit in arm swing or the like. Therefore, by obtaining the representative value of the elbow angle in one cycle (or more) of the arm swing, the electronic device 1 can identify the elbow angle θe more accurately.

Further, the electronic device 1 may include the measurer 18, which measures the three-dimensional acceleration. This enables the electronic device 1 to obtain the amount of the arm swing on the basis of the acceleration measured by itself with simple and easy processes and provide it to the user. Thus, the electronic device 1 can provide the user with appropriate arm swing information while being lightweight with a gyro sensor or the like eliminated (not included).

Further, the arm swing analysis method that is performed by the CPU 11 (at least one processor) of the above embodiments includes identifying a cyclic change related to the arm swing of a user from the three-dimensional acceleration measured by the measurer measures the three-dimensional acceleration. The arm swing analysis method includes defining a two-dimensional plane containing the plane of the arm swing of the user from the three-dimensional acceleration measured at a wrist (Bw) of the user, detecting the cyclic change as a motion on an arc centered at a shoulder (Bs) of the user in the two-dimensional plane, obtaining, from the three-dimensional acceleration, the direction of the line segment connecting the wrist Bw and the shoulder Bs, and calculating (obtaining) the elbow angle θe based on the line segment.

This arm swing analysis method can easily obtain the relationship between the z-axis direction, which is the average orientation of the arm, and the R-axis direction, which is the direction from the wrist Bw to the shoulder Bs, and, by extension, obtain the elbow angle θe. Thus, the arm swing analysis method can identify the user's arm state during the arm swing more easily.

Further, by installing the program 131 of the above embodiments into a computer to cause the computer to execute the program 131, the elbow angle θe can be calculated (obtained) easily from the measurement result of the three-dimensional acceleration by the measurer 18. The program 131 can provide the user with appropriate information on the elbow angle θe at low cost. This enables the computer to evaluate the user's form, posture and so forth in walking or running more accurately.

The above embodiments are merely examples, and various modifications can be made.

For example, in the above, the elbow angle θe is calculated to calculate the arm swing angle φ0, but not limited thereto. The elbow angle θe may be obtained to obtain another index for evaluating the posture or the like in running, jogging or the like. Alternatively, the elbow angle θe itself may be used as an index for evaluating the posture.

Further, there is a case where a user is not moving although he/she is swinging his/her arms. For example, the user may be checking his/her arm swing form while standing still or stepping in place. The CPU 11 may make a conditional determination to detect the arm swing even if the user is not running/walking.

Further, in the above, the median value or the like of the elbow angles φe is obtained as the representative value of the elbow angle θe in one cycle of the arm swing, but the representative value may not be obtained from a plurality of calculated values. The elbow angle θe at a certain point of time may be output as it is. In this case, the certain point of time may be a specific timing in one cycle, such as a timing at which the z-axis is parallel to the vertical direction, or a turn timing. Alternatively, the certain point of time may be the center timing of all the timings in one cycle. Still alternatively, the elbow angle θe may be calculated intermittently from two or more measured data of all the measured data in one cycle, and the average value or the like of these may be used as the representative value.

Further, unlike the above, the LPF may not be used to extract the DC component from the acceleration in one cycle of the arm swing. Any other technique may be used therefor.

Further, in the above, the upper arm length Lu and the forearm length Lf are determined on the basis of the height. As described above, the lengths Lu, Lf may be determined by additionally taking the age, gender, race, weight, foot size and/or the like into account. Alternatively, the lengths Lu, Lf may be roughly classified on the basis of some or all of the age, gender, race, weight, foot size and/or the like without taking the height into account, or more simply, may be treated as constants.

Further, in the modification of the first embodiment, the inclination of the line segment connecting the shoulder Bs and the wrist Bw from the vertical direction may not be obtained on the basis of the gravitational acceleration, but obtained by another method.

Further, the electronic device 1 may perform the minimal display only. Except for the notification (notifying action) by the notifier 19, display of the detailed graphs, history (records) and so forth mentioned above may be performed by the external electronic device 4 only.

Further, in the above, the electronic device 1 is in the shape of an electronic wristwatch, but not limited thereto. For example, the electronic device 1 may be in the shape of a simple belt. Further, as far as the electronic device 1 can be fixed to a wrist, it may not be fixed by using a band or the like that is wrapped around the arm (wrist).

Further, the arm swing analysis and the notifying action may not be performed by the electronic device 1. The measured data and some or all of the analysis data may be transmitted to the electronic device 4, and undone analysis if any and the notifying action may be performed by the electronic device 4.

Further, in the above, the electronic device 1 includes both the CPU 11 and the measurer 18, but not limited thereto. For example, an external acceleration sensor may be attached to the electronic device 1 that includes a control device.

Further, the electronic device 7 may be mounted on, instead of the waist, any other position except an arm(s) (i.e., except positions between a shoulder and fingertips). In this case, the electronic device 7 is preferably mounted on a position as close as possible to the trunk, or on a leg that is likely to be influenced by its landing on the ground.

Further, in the above, as a computer-readable storage medium storing the program 131 for the arm swing index calculation control, the memory 13 composed of a nonvolatile memory, such as a flash memory, is used, but the computer-readable storage medium is not limited thereto. As the computer-readable storage medium, another type of nonvolatile memory, such as an MRAM or an HDD, or a portable storage medium, such as a CD-ROM or a DVD, can also be used. Further, as a medium to provide data of the program(s) of the present disclosure via a communication line, a carrier wave is also applicable to the present disclosure.

The configurations, components, processes' contents and procedures, and so forth detailed in the above embodiments can be changed as appropriate without departing from the scope of the present disclosure. The scope of the present disclosure includes the scope of claims and the scope of their equivalents.

1. An electronic device comprising at least one processor that identifies a cyclic change related to an arm swing of a user from a three-dimensional acceleration measured by a measurer,
wherein the at least one processor
defines a two-dimensional plane containing a plane of the arm swing of the user from the three-dimensional acceleration measured at a wrist of the user,
detects the cyclic change as a motion on an arc centered at a shoulder of the user in the two-dimensional plane,
obtains, from the three-dimensional acceleration, a direction of a line segment connecting the wrist and the shoulder, and
obtains a bending angle of an elbow of the user based on the line segment.

2. The electronic device according to claim 1, wherein the motion is a reciprocating motion.

3. The electronic device according to claim 2, wherein the at least one processor
identifies an acceleration in the two-dimensional plane based on the three-dimensional acceleration measured during the arm swing,
extracts a direct-current component from the acceleration in the two-dimensional plane to identify the direction of the line segment,
obtains a first angle formed by the line segment and a direction from the wrist to the elbow of the user, and
obtains the bending angle of the elbow based on the obtained first angle.

4. The electronic device according to claim 3, wherein the at least one processor obtains the bending angle of the elbow based on a length of an arm of the user and the first angle.

5. The electronic device according to claim 1, wherein the at least one processor
obtains the bending angle of the elbow from each of the three-dimensional acceleration measured in a period of one or more cycles of the arm swing, thereby obtaining a plurality of bending angles of the elbow, and
determines a representative value of the bending angle of the elbow from the obtained plurality of bending angles of the elbow.

6. The electronic device according to claim 1, further comprising the measurer that measures the three-dimensional acceleration.

7. The electronic device according to claim 2, further comprising the measurer that measures the three-dimensional acceleration.

8. The electronic device according to claim 3, further comprising the measurer that measures the three-dimensional acceleration.

9. The electronic device according to claim 4, further comprising the measurer that measures the three-dimensional acceleration.

10. The electronic device according to claim 5, further comprising the measurer that measures the three-dimensional acceleration.

11. An arm swing analysis method that is performed by at least one processor, comprising identifying a cyclic change related to an arm swing of a user from a three-dimensional acceleration measured by a measurer,
wherein the identifying includes:
defining a two-dimensional plane containing a plane of the arm swing of the user from the three-dimensional acceleration measured at a wrist of the user;
detecting the cyclic change as a motion on an arc centered at a shoulder of the user in the two-dimensional plane;
obtaining, from the three-dimensional acceleration, a direction of a line segment connecting the wrist and the shoulder; and
obtaining a bending angle of an elbow of the user based on the line segment.

12. The arm swing analysis method according to claim 11, wherein the motion is a reciprocating motion.

13. The arm swing analysis method according to claim 12, wherein the identifying includes:
identifying an acceleration in the two-dimensional plane based on the three-dimensional acceleration measured during the arm swing;
extracting a direct-current component from the acceleration in the two-dimensional plane to identify the direction of the line segment;
obtaining a first angle formed by the line segment and a direction from the wrist to the elbow of the user; and
obtaining the bending angle of the elbow based on the obtained first angle.

14. The arm swing analysis method according to claim 13, wherein the obtaining the bending angle of the elbow includes obtaining the bending angle of the elbow based on a length of an arm of the user and the first angle.

15. The arm swing analysis method according to claim 11, wherein the identifying includes:
obtaining the bending angle of the elbow from each of the three-dimensional acceleration measured in a period of one or more cycles of the arm swing, thereby obtaining a plurality of bending angles of the elbow, and
determining a representative value of the bending angle of the elbow from the obtained plurality of bending angles of the elbow.

16. A non-transitory computer-readable storage medium storing a program that causes a computer to perform identifying a cyclic change related to an arm swing of a user from a three-dimensional acceleration measured by a measurer,
wherein the identifying includes:
defining a two-dimensional plane containing a plane of the arm swing of the user from the three-dimensional acceleration measured at a wrist of the user;
detecting the cyclic change as a motion on an arc centered at a shoulder of the user in the two-dimensional plane;
obtaining, from the three-dimensional acceleration, a direction of a line segment connecting the wrist and the shoulder; and
obtaining a bending angle of an elbow of the user based on the line segment.
